(19) United States
(12) Patent Application Publication
(10) Pub. No.: US 2024/0191171 A1
(43) Pub. Date: Jun. 13, 2024

(54) BIOREACTOR SYSTEM AND CELL CULTURE METHOD USING THE SAME

(71) Applicant: Pensees Inc., Seoul (KR)

(72) Inventors: Sung Jun LEE, Gimhae-si (KR); Chang Ve CHEAH, Seoul (KR)

(73) Assignee: Pensees Inc., Seoul (KR)

(21) Appl. No.: 18/152,229

(22) Filed: Jan. 10, 2023

(30) Foreign Application Priority Data
Dec. 7, 2022 (KR) 10-2022-0170025

(51) Int. Cl. C12M 1/04; C12M 1/00; C12M 1/34

(52) U.S. Cl. CPC C12M 27/04; C12M 29/18; C12M 41/26; C12M 41/34

(57) ABSTRACT

A bioreactor system includes a bioreaction unit configured to accommodate a culture medium, and a treatment unit configured to recycle the culture medium transferred from the bioreaction unit, wherein the treatment unit includes a treatment tank which accommodates the culture medium transferred from the bioreaction unit, a culture medium exchange part configured to exchange the culture medium between the bioreaction unit and the treatment tank, and a waste product removal part configured to remove waste products contained in the culture medium accommodated in the treatment tank.

### CROSS-REFERENCE TO RELATED APPLICATIONS

This application claims the priority of Korean Patent Application No. 10-2022-0170025 filed on Dec. 7, 2022, in the Korean Intellectual Property Office, the disclosure of which is incorporated herein by reference.

### BACKGROUND OF THE INVENTION

### Field of the Invention

The present invention relates to a bioreactor system and a cell culture method using the same.

### Description of the Related Art

A bioreactor refers to a mechanical device manufactured and designed in engineering in order to make a biological activation environment. The bioreactor is like a vessel that causes a chemical reaction of organic matter extracted from living organisms, or biochemically activated substances. This process may be aerobic or anaerobic. On the basis of the operational mode, the bioreactors are classified into batch, fed-batch, and continuous reactors (e.g., continuous stirred reactor, etc.). An example of the continuous fermentor is chemostat.

The bioreactor can be used for cell culture. When a culture medium and cells are introduced into the bioreactor, the bioreactor may stir the culture medium containing the cells to induce the growth of the cells through the exchange of materials.

As the cells are cultured in the bioreactor, waste products, such as lactic acid and ammonia produced as by-products of cell growth, are accumulated in the culture medium. The toxicity of these waste products may interfere with cell culture. In addition, the state of the culture medium in the bioreactor may change to a state that is not suitable for cell culture, such as depletion of nutrients in the culture medium or change in composition of the dissolved gas.

However, if the culture medium is completely replaced in order to remove waste products, the cells may not be smoothly cultured during the replacement process. In addition, the cells may be lost during the replacement process.

### SUMMARY OF THE INVENTION

An aspect of the present invention provides a bioreactor system which can recycle a culture medium without interfering with cell culture, and a cell culture method using the bioreactor system.

According to an aspect of the present invention, there is provided a bioreactor system including: a bioreaction unit configured to accommodate a culture medium; and a treatment unit configured to recycle the culture medium transferred from the bioreaction unit, wherein the treatment unit includes a treatment tank which accommodates the culture medium transferred from the bioreaction unit, a culture medium exchange part configured to exchange the culture medium between the bioreaction unit and the treatment tank, and a waste product removal part configured to remove waste products contained in the culture medium accommodated in the treatment tank.

According to another aspect of the present invention, there is provided a cell culture method including: transferring a culture medium accommodated in a reaction tank to a treatment tank; removing waste products contained in the culture medium accommodated in the treatment tank; and transferring, to the reaction tank, the culture medium from which the waste products are removed.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a view schematically illustrating a bioreactor system according to an exemplary embodiment of the present invention;

FIG. 2 is a view illustrating a state in which the bioreactor system according to an exemplary embodiment of the present invention is realized;

FIG. 3 is a view illustrating a removal member of a dialyzer according to an exemplary embodiment of the present invention;

FIG. 4 is a view schematically illustrating a bioreactor system according to another exemplary embodiment of the present invention; and

FIG. 5 is a view illustrating a state in which the bioreactor system according to another exemplary embodiment of the present invention is realized.

### DETAILED DESCRIPTION OF THE PREFERRED EMBODIMENT

Exemplary embodiments of the present invention will now be described in detail with reference to the accompanying drawings. In adding reference numerals to components in each drawing, the same components will be designated by the same reference numerals, if possible, although they are shown in different drawings. Further, in the following description of the exemplary embodiments of the present invention, a detailed description of well-known configurations or functions incorporated herein will be omitted when it is determined that the description may prevent the exemplary embodiments of the present invention from being understood.

In addition, terms, such as first, second, A, B, (a), and (b) may be used herein when describing the components of exemplary embodiments of the present invention. These terms are merely used to distinguish one component from other components, and properties, an order, a sequence, or the like of the corresponding component are not limited by the terms. If a component is described as ‘connected’, ‘coupled’, or ‘linked’ to another component, one of ordinary skill in the art would understand the components may be not only directly ‘connected’, ‘coupled’, or ‘linked’ but may also be indirectly ‘connected’, ‘coupled’, or ‘linked’ via a third component.

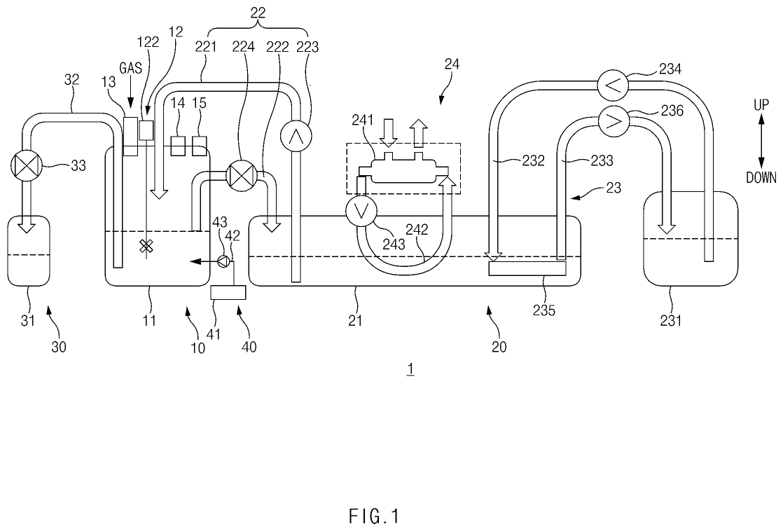

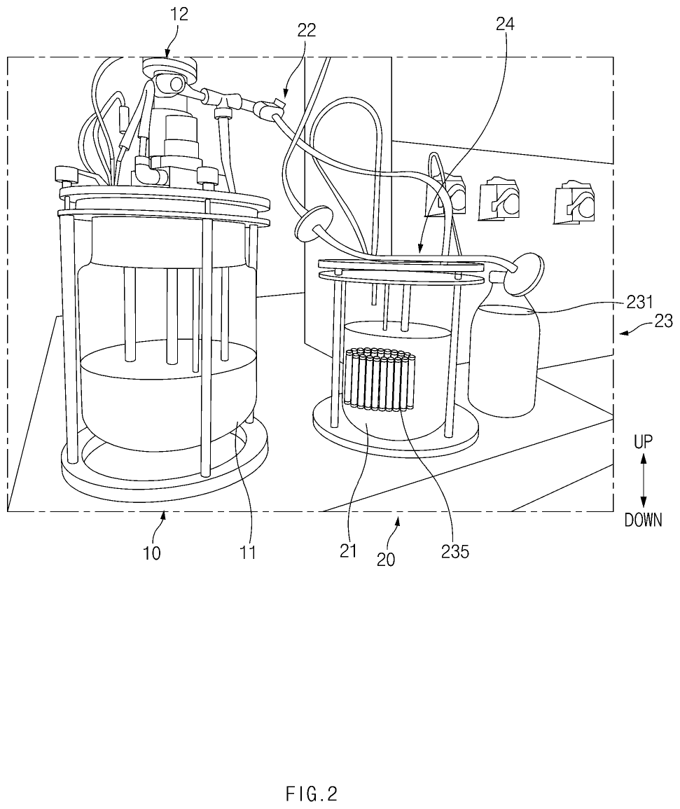

FIG. 1 is a view schematically illustrating a bioreactor system 1 according to an exemplary embodiment of the present invention. FIG. 2 is a view illustrating a state in which the bioreactor system 1 according to an exemplary embodiment of the present invention is realized.

Referring to FIGS. 1 and 2, the bioreactor system 1 according to an exemplary embodiment of the present invention includes a bioreaction unit 10 and a treatment unit 20. The bioreactor system 1 according to an exemplary embodiment of the present invention may include a processor. The bioreactor system 1 according to an exemplary embodiment of the present invention may include a harvesting unit 30. The bioreactor system 1 according to an exemplary embodiment of the present invention may include a feeding unit 40.

### Bioreaction Unit 10

The bioreaction unit 10 may accommodate a culture medium. The bioreaction unit 10 includes a reaction tank 11 capable of accommodating the culture medium therein. The reaction tank 11 may accommodate cells for being cultured with the culture medium.

The bioreaction unit 10 may include an agitator 12. The agitator 12 may stir the culture medium accommodated in the reaction tank 11. Therefore, the agitator 12 may include, in the reaction tank 11, a blade for stirring the culture medium and a shaft to which the blade is coupled at one end, and may include a stirring driver connected to the other end of the shaft to rotate the shaft. The stirring driver may be disposed outside the reaction tank 11. That is, the agitator 12 may be coupled to the reaction tank 11 while passing through the reaction tank 11. The agitator 12 may pass through the top of the reaction tank 11. The stirring driver may be a motor capable of generating a rotational driving force. By the stirring operation of the agitator 12, the culture medium and the cells may be well mixed to promote cell growth.

The bioreaction unit 10 may include a gas inlet part 13. The gas inlet part 13 may be configured to introduce a gas into the reaction tank 11. That is, the gas inlet part 13 may be coupled to the reaction tank 11 while passing through the reaction tank 11. The gas inlet part 13 may pass through the top of the reaction tank 11. The gas inlet part 13 may include a gas inlet part conduit which allows the inside and the outside of the reaction tank 11 to communicate with each other, and may include a gas inlet part valve for opening and closing the gas inlet part conduit. The gas inlet part 13 may include a gas inlet part pump that is connected to the gas inlet part conduit in order to pump the gas into the reaction tank 11.

The bioreaction unit 10 may include a pH obtaining part 14 capable of obtaining a pH (hydrogen-ion concentration) of the culture medium accommodated in the reaction tank 11. The pH obtaining part 14 may be coupled to the reaction tank 11. The pH obtaining part 14 may be a pH sensor. The pH sensor may be a sensor using a semiconductor or may be a sensor composed of an electrode, and a type thereof is not limited thereto.

The bioreaction unit 10 may include an oxygen amount obtaining part 15 capable of obtaining a dissolved oxygen (DO) of the culture medium accommodated in the reaction tank 11. The oxygen amount obtaining part 15 may be coupled to the reaction tank 11. The oxygen amount obtaining part 15 may be a dissolved oxygen sensor. The dissolved oxygen sensor may be a conductivity sensor, a specific resistance sensor, or an optical sensor, but a type thereof is not limited thereto.

### Harvesting Unit 30

The harvesting unit 30 may be connected to the bioreaction unit 10 to receive the culture medium accommodated in the bioreaction unit 10. The harvesting unit 30 may receive the cells cultured in the bioreactor 10 together with the culture medium. Accordingly, the harvesting unit 30 may include a harvesting tank 31 for storing the culture medium and the cells, and a harvesting conduit 32 having one end connected to the reaction tank 11 and the other end connected to the harvesting tank 31 to transfer the culture medium and the cells from the reaction tank 11 to the harvesting tank 31. One end of the harvesting conduit 32 located in the reaction tank 11 is disposed adjacent to the bottom of the reaction tank 11 so that the cells settled in the bottom may be easily harvested. The harvesting unit 30 may include a harvesting valve 33 for opening and closing the harvesting conduit 32.

### Feeding Unit 40

The feeding unit 40 may supply nutrients to the culture medium. As the cells are cultured in the reaction tank 11, nutrients for cell growth contained in the culture medium are depleted. Therefore, it is necessary to continuously supply nutrients by introducing a fresh culture medium. However, due to the limitation of the size of the bioreactor, the culture medium cannot be introduced indefinitely. Accordingly, nutrients such as proteins may be supplied to the reaction tank 11 through the feeding unit 40.

The feeding unit 40 may include a nutrient tank 41 for storing nutrients, and a nutrient conduit 42 having one end connected to the reaction tank 11 and the other end connected to the nutrient tank 41 to transfer the nutrients from the nutrient tank 41 to the reaction tank 11. The feeding unit 40 may include a nutrient pump 43 for pumping the nutrients stored in the nutrient tank 41 into the reaction tank 11 through the nutrient conduit 42.

The exemplary embodiment of the present invention describes that the feeding unit 40 is connected to the reaction tank 11, but the feeding unit 40 may be connected to the treatment unit 20.

### Treatment Unit 20

The treatment unit 20 may recycle the culture medium received from the bioreaction unit 10. Recycling the culture medium means changing the state of the culture medium to a state suitable for cell culture. Accordingly, for the recycling of the culture medium, waste products contained in the culture medium may be removed, nutrients may be supplied to the culture medium, or a gas exchange may be performed in order to make the dissolved gas component of the culture medium suitable for cell growth.

The treatment unit 20 may include a treatment tank 21. The treatment tank 21 may accommodate the culture medium, inside the treatment tank 21, received from the bioreaction unit 10. A fresh culture medium may be supplied to the treatment tank 21 in a device, etc. other than the reaction tank 11.

The treatment unit 20 may include a culture medium exchange part 22. The culture medium exchange part 22 may exchange the culture medium between the bioreaction unit 10 and the treatment tank 21. The culture medium exchange part 22 may include exchange conduits 221 and 222 for this exchange operation. The exchange conduits 221 and 222 may include a first exchange conduit 221 and a second exchange conduit 222. The first exchange conduit 221 may transfer the culture medium from the treatment tank 21 to the reaction tank 11. The second exchange conduit 222 may transfer the culture medium from the reaction tank 11 to the treatment tank 21.

The culture medium exchange part 22 may be operated to transfer the culture medium in the reaction tank 11 to the treatment tank 21 so that the distance between the lower end of the second exchange conduit 222 and the bottom of the reaction tank 11 is equal to or lower than the level of the culture medium accommodated in the reaction tank 11 on the basis of the vertical direction. Accordingly, the second exchange conduit 222 may transfer the supernatant containing the waste products, which are small molecule, among the culture medium in the reaction tank 11 to the treatment tank 21, and may prevent the cells or nutrients that are relatively heavy and thus sink in the reaction tank 11 from being transferred to the treatment tank 21.

The culture medium exchange part 22 may include an exchange pump 223. The exchange pump 223 may be disposed in the first exchange conduit 221 so as to pump the culture medium contained in the treatment tank 21 into the reaction tank 11. The culture medium exchange part 22 may include an exchange valve 224 disposed in the second exchange conduit 222 so as to control opening and closing of the second exchange conduit 222.

The treatment unit 20 may include an aeration part 24. The aeration part 24 may provide air to the culture medium accommodated in the treatment tank 21. The aeration part 24 may provide other gases in addition to air to the culture medium accommodated in the treatment tank 21, and may receive the gases contained in the culture medium and discharge the gases to the outside. In order to enable such an operation without generation of bubbles, the aeration part 24 may be an aeration device using a hollow fiber membrane, etc. permeable to oxygen, but a type thereof is not limited thereto.

The aeration part 24 may include a gas inlet part and outlet part 241 through which a gas flows in and out, and an aeration contact tube 242 which is connected to the gas inlet part and outlet part 241, receives the gas or transfers the gas to the gas inlet part and outlet part 241, and is in contact with the culture medium in the treatment tank 21. As the gas flows into the aeration contact tube 242 and the culture medium flows out of the aeration contact tube 242, the gas exchange between the culture medium and the inside of the aeration contact tube 242 may occur. The aeration contact tube 242 may be formed of the above-described membrane. The aeration part 24 may include an aeration pump 243 for pumping gas in one direction through the aeration contact tube 242.

The treatment unit 20 may include a waste product removal part. The waste product removal part may remove waste products contained in the culture medium accommodated in the treatment tank 21. The dialyzer 23 may be used as a waste product removal part. Hereinafter, the waste product removal part will be described as the dialyzer 23, but a type thereof is not limited thereto and various means may be used as long as it may remove the waste products from the culture medium.

The dialyzer 23 may include a buffer tank 231. The buffer tank 231 may accommodate a buffer solution therein. The buffer solution may be a solution having a lower concentration than the culture medium. The dialyzer 23 may include a removal module. The removal module may be partially positioned in the treatment tank 21 so that the buffer solution accommodated in the buffer tank 231 is circulated through the inside of the removal module and waste products are transferred from the culture medium accommodated in the treatment tank 21 to the buffer solution. The removal module may include a removal member 235 which is positioned inside the treatment tank 21 and has a semi-permeable membrane.

The removal module may include buffer transfer conduits 232 and 233. The buffer delivery conduits 232 and 233 may connect the buffer tank 231 and a plurality of dialysis tubes 2353 so that the buffer solution accommodated in the buffer tank 231 flows in the plurality of dialysis tubes 2353 included in the removal member 235. The buffer transfer conduits 232 and 233 may include a first buffer transfer conduit 232 and a second buffer transfer conduit 233. The first buffer transfer conduit 232 may transfer the buffer solution from the treatment tank 21 to the removal member 235. The second buffer transfer conduit 233 may transfer the buffer solution from the removal member 235 to the treatment tank 21.

A buffer flow path, which is a path through which the buffer solution circulates, may be formed by the buffer transfer conduits 232 and 233 and the removal member 235. In order to flow the buffer solution through the buffer flow path, the dialyzer 23 may include pump parts 234 and 236. The pump parts 234 and 236 may be disposed in the buffer transfer conduits 232 and 233 so as to pump the buffer solution to the dialysis tube 2353. The pump parts 234 and 236 may include a peristaltic pump, but a type thereof is not limited thereto.

The pump parts 234 and 236 may include a first pump 234 and a second pump 236. The first pump 234 and the second pump 236 may be peristaltic pumps. The first pump 234 and the second pump 236 may be disposed adjacent to the inlet and the outlet of the buffer flow path, respectively. The first pump 234 may be disposed in the first buffer transfer conduit 232, and the second pump 236 may be disposed in the second buffer transfer conduit 233. The first pump 234 and the second pump 236 may be disposed at both ends of the buffer flow path to maintain, within a certain range, the amount of the buffer solution positioned in the buffer flow path, thereby preventing the culture medium from flowing out of the treatment tank 21 to the removal member 235 due to the reverse osmosis phenomenon.

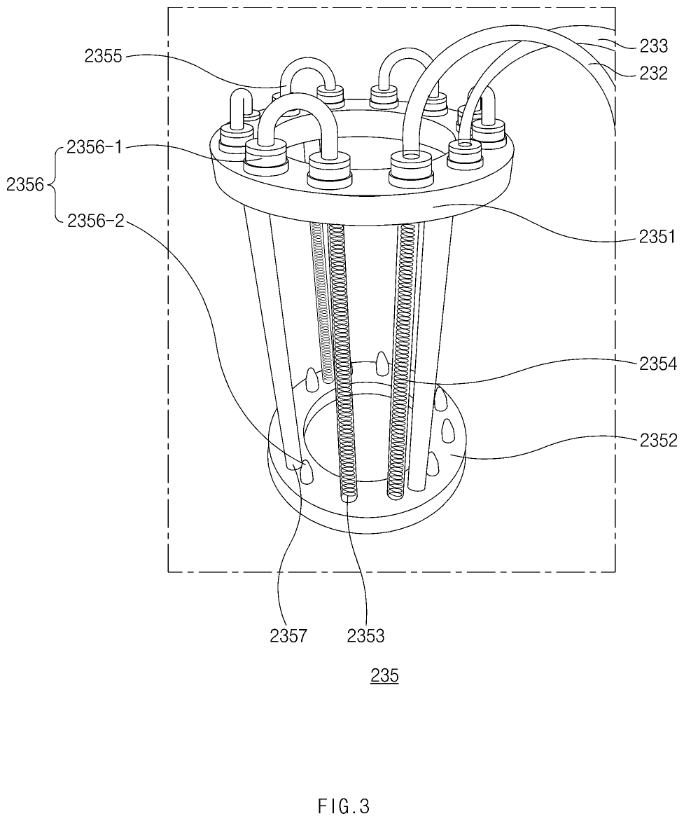

FIG. 3 is a view illustrating the removal member 235 of the dialyzer 23 according to an exemplary embodiment of the present invention.

The dialyzer 23 may include a removal member 235. The removal member 235 may be positioned inside the treatment tank 21 and immersed in the culture medium. The buffer solution may flow into the removal member 235, and the culture medium may flow out of the removal member 235 so that waste products contained in the culture medium may be transferred to the buffer solution via the removal member 235.

The removal member 235 may include a pair of frames 2351 and 2352. The pair of frames are composed of a first frame 2351 and a second frame 2352. The pair of frames 2351 and 2352 may each be formed in an annular shape. The pair of frames 2351 and 2352 may be disposed to be spaced apart from each other in a direction orthogonal to the ring. In an exemplary embodiment of the present invention, it is described that the pair of frames 2351 and 2352 are formed in an annular shape orthogonal to the vertical direction and are spaced apart from each other in the vertical direction, but the shape and arrangement thereof are not limited thereto.

If the frames 2351 and 2352 are formed in an annular shape, thus the culture medium may flow through the centers of the frames 2351 and 2352, and the agitator and the removal member 235 are disposed in the same space, the agitator may be disposed through the center of the frames 2351 and 2352.

The removal member 235 may include a column 2357. The column 2357 may extend in one direction, and the both ends of the column 2357 may be connected to the pair of frames 2351 and 2352, respectively. Accordingly, the column 2357 may connect the pair of frames 2351 and 2352 and maintain the overall shape of the removal member 235. The column 2357 may be composed in plural.

The removal member 235 may include an adapter 2356. The adapter 2356 may be coupled to the frames 2351 and 2352 while passing through the frames 2351 and 2352. The adapter 2356 may allow the dialysis tube 2353 and the connection tube 2355 to communicate with each other. One end of the adapter 2356 may be coupled to the dialysis tube 2353, and the other end thereof may be coupled to the connection tube 2355. Here, one end of the adapter 2356 may be disposed between the pair of frames 2351 and 2352, and the other end of the adapter 2356 may be disposed outside the pair of frames 2351 and 2352.

One end of the adapter 2356 may be an insertion end 2356-2. The insertion end 2356-2 may be inserted into the dialysis tube 2353 and coupled to the dialysis tube 2353. The insertion end 2356-2 may have a nipple shape. The other end of the adapter 2356 may be a receiving end 2356-1. The connection tube 2355 may be inserted into the receiving end 2356-1, and the receiving end 2356-1 and the connection tube 2355 may be coupled to each other. Accordingly, the receiving end 2356-1 may be formed in a ring shape into which the connection tube 2355 may be inserted. As illustrated, through the receiving end 2356-1, the first buffer transfer conduit 232 and the second buffer transfer conduit 233 may be connected to the inlet and the outlet of the dialysis flow path, respectively, at the end of the dialysis flow path which will be described later.

A plurality of the adapters 2356 may be disposed to be spaced apart in the circumferential direction of the frames 2351 and 2352. The adapter 2356 may be formed of an elastic material and may maintain airtightness at the boundary when coupled to the connection tube 2355 and the dialysis tube 2353.

The removal member 235 may include a plurality of dialysis tubes 2353. The dialysis tube 2353 may be a semi-permeable tube made of a semi-permeable film. Therefore, the material exchange between the external culture medium and the internal buffer solution may be performed through the dialysis tube 2353. The pressure in the dialysis tube 2353 may be maintained to be similar to the treatment tank 21 using the pump parts 234 and 236, and the concentration of the buffer solution may be maintained to be significantly lower than the culture medium, so that the waste products may be moved from the culture medium to the buffer solution by diffusion due to a concentration difference in which the solute moves, rather than reverse osmosis.

A plurality of dialysis tubes 2353 may be coupled to the pair of frames 2351 and 2352. The plurality of dialysis tubes 2353 may be coupled to the frames 2351 and 2352 via the adapter 2356. The dialysis tube 2353 may extend in one direction, and both ends thereof may be coupled to the insertion ends 2356-2 of the adapter 2356, respectively. The direction in which the dialysis tube 2353 extends is a vertical direction in an exemplary embodiment of the present invention, but the direction is not limited thereto. The plurality of dialysis tubes 2353 may be disposed to be spaced apart from each other in the circumferential directions of the frames 2351 and 2352.

The removal member 235 may include a plurality of connection tubes 2355 connecting the plurality of dialysis tubes 2353. Each connection tube 2355 may connect adjacent dialysis tubes 2353 to each other among the plurality of dialysis tubes 2353. Both ends of the connection tube 2355 may be coupled to receiving ends 2356-1 of different adapters 2356, respectively. To connect each dialysis tube 2353, the connection tube 2355 may have a U-shaped form. For easy deformation, the connection tube 2355 may not be a semi-permeable tube, but a tube having a general soft material.

The plurality of dialysis tubes 2353 may be positioned between the pair of frames 2351 and 2352, and the plurality of connection tubes 2355 may be positioned outside the pair of frames 2351 and 2352 opposite to the plurality of dialysis tubes 2353 with respect to the pair of frames 2351 and 2352.

The plurality of connection tubes 2355 and the plurality of dialysis tubes 2353 may be connected to each other to form a dialysis flow path, which is a part of the buffer flow path, as a series flow path. Here, the series flow path means that the fluid does not separate but flows along one path. That is, the inlet and the outlet of at least some of the dialysis tubes 2353 are not common, and the outlet of one dialysis tube 2353 may be connected to the inlet of the other dialysis tube 2353 through a connection flow path.

The dialysis flow path, which is a series flow path, may extend tortuously in the circumferential direction of the frames 2351 and 2352. That is, for example, the dialysis flow path may be formed in such a manner that after the buffer solution flows from the first frame 2351 to the second frame 2352 through the dialysis flow path, the flow direction of the buffer solution may be reversed by the connection flow path, and the buffer solution flows from the second frame 2352 to the first frame 2351 through the dialysis flow path.

The removal member 235 may include a mixer 2354. The mixer 2354 may be disposed inside the dialysis tube 2353. The mixer 2354 may extend in a direction in which the dialysis tube 2353 extends, and may have a spiral structure. The mixer 2354 may prevent the dialysis tube 2353 from being dented, and may allow material exchange to occur for a longer period of time as the buffer solution flowing in the dialysis tube 2353 passes through the dialysis tube 2353 along a spiral path.

### Processor

The processor is configured to control the components of the bioreactor system 1. The processor is a component including an element capable of performing a logical operation for performing a control command, and may include a central processing unit (CPU) and the like. The processor may be connected with various components to perform control by transmitting signals according to control commands to respective components, and may be connected with various sensors or obtaining parts to receive obtained information in the form of a signal. Therefore, in an exemplary embodiment of the present invention, the processor may be electrically connected with various components included in the bioreactor system 1. The processor may be electrically connected with the respective components. Accordingly, a communication module connected with the processor through a conductive wire or being able to wirelessly communicate with the processor may be further provided, such that the processor may communicate with the respective components. Here, the electrical connection includes not only physical connection with each other to allow an electric current to flow, but also communicatable connection with each other so as to transmit and receive control signals with each other.

The bioreactor system 1 may further include a storage medium, and control commanders performed by the processor may be stored in the storage medium and utilized. The storage medium may be a device such as a hard disk drive (HDD), a solid state drive (SSD), a server, a volatile medium, and a non-volatile medium, but a type thereof is not limited thereto. In addition, the storage medium may further store data or the like necessary for the processor to perform tasks.

Hereinafter, a cell culture method using the bioreactor system 1 according to an exemplary embodiment of the present invention will be described.

The processor may operate the agitator 12. The processor may operate the agitator 12 so that the culture medium is stirred. The processor may control the culture medium exchange part 22 in order to move the culture medium from the treatment tank 21 to the reaction tank 11. That is, stirring the culture medium accommodated in the reaction tank 11 may be performed. Delivering the culture medium, from which the waste products have been removed, in the treatment tank 21 to the reaction tank 11 may be performed, which may be the delivering of the culture medium to the reaction tank 11 while the culture medium accommodated in the reaction tank 11 is stirred.

The processor may operate the gas inlet part 13 so that the culture medium moves through the second exchange conduit 222. In addition, the processor may open the exchange valve 224 so that the culture medium moves through the second exchange conduit 222. A gas is introduced into the reaction tank 11 by the operation of the gas inlet part 13, and the water level rises as the pressure of the reaction tank 11 increases so that the supernatant of the culture medium in the reaction tank 11 may be transferred to the treatment tank 21 through the second exchange conduit 222. The operation of the gas inlet part 13 may be performed after the processor stops the operation of the agitator 12, and after the operation of the agitator 12 is stopped, a predetermined waiting time passes, and then the operation of the gas inlet part 13 may be performed.

That is, transferring the culture medium accommodated in the reaction tank 11 to the treatment tank 21 may include introducing the gas into the reaction tank 11 and transferring the culture medium from the reaction tank 11 to the treatment tank 21 through the exchange conduits 221 and 222 by the increased pressure. In addition, before the culture medium accommodated in the reaction tank 11 is transferred to the treatment tank 21, stopping the stirring of the culture medium accommodated in the reaction tank 11 may be performed. After the operation of the agitator 12 is stopped, a predetermined waiting time has passed, and then the gas inlet part 13 may be operated to perform the transferring of the culture medium to the treatment tank 21.

As described above, after the stirring is stopped, the culture medium is moved after the waiting time, and the supernatant including the waste product layer formed after the cells floated by the stirring and the nutrients are sufficiently settled may be transferred to the treatment tank 21.

The processor may determine whether the culture medium exchange is necessary. If it is determined that culture medium exchange is necessary, the processor may control the culture medium exchange part 22 so that the culture medium is exchanged. In addition, if it is determined that culture medium exchange is necessary, the processor may operate the waste product removal part or the aeration part 24.

The processor may determine whether the culture medium exchange is necessary on the basis of the pH value of the culture medium in the reaction tank 11 obtained by the pH obtaining part 14, and thus control the operation of the culture medium exchange part 22 or the waste product removal part. The processor may determine whether the pH value obtained by the pH obtaining part 14 is within a predetermined allowable pH range, and may determine that the culture medium exchange is necessary if the pH value is out of the range.

The processor may determine whether the culture medium exchange is necessary on the basis of the dissolved oxygen amount value of the culture medium in the reaction tank 11 obtained by the oxygen amount obtaining part 15, and thus control the operation of the culture medium exchange part 22 or the waste product removal part. The processor may determine whether the dissolved oxygen amount value obtained by the oxygen amount obtaining part 15 is within a predetermined allowable oxygen amount range, and may determine that the culture medium exchange is necessary if the dissolved oxygen amount value is out of the range.

The processor may remove waste products by operating the waste product removal part. The processor operates the dialyzer 23, which is a waste product removal part, so that the buffer solution circulates in the removal module and the waste products in the treatment tank 21 may be removed. That is, removing the waste products contained in the culture medium accommodated in the treatment tank 21 may be performed. This step may be transferring the waste products from the culture medium to the buffer solution using a semi-permeable membrane.

The culture medium from which the waste products have been removed may be transferred to the reaction tank 11 again through the culture medium exchange part 22. Therefore, the recycled culture medium may be continuously supplied to the reaction tank 11 without stopping the cell culture.

### Another Exemplary Embodiment

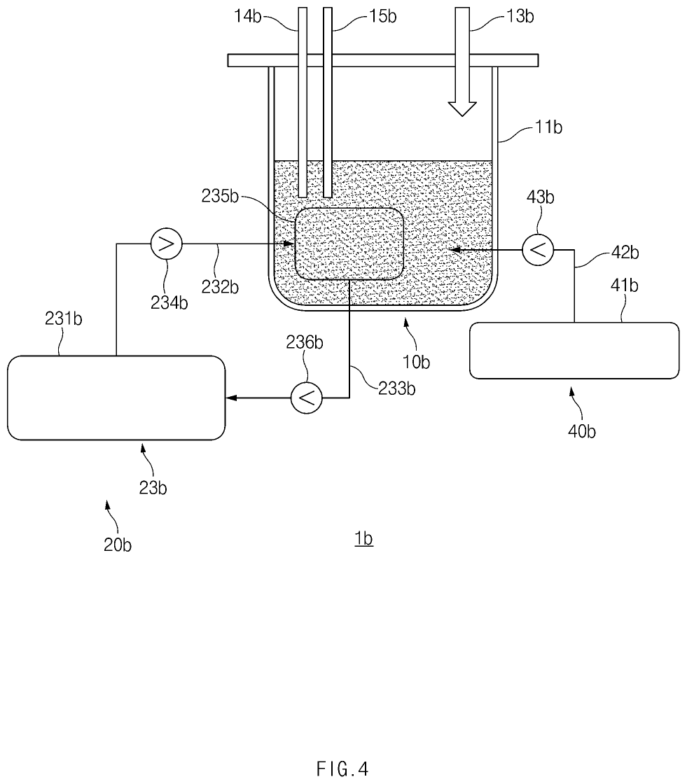

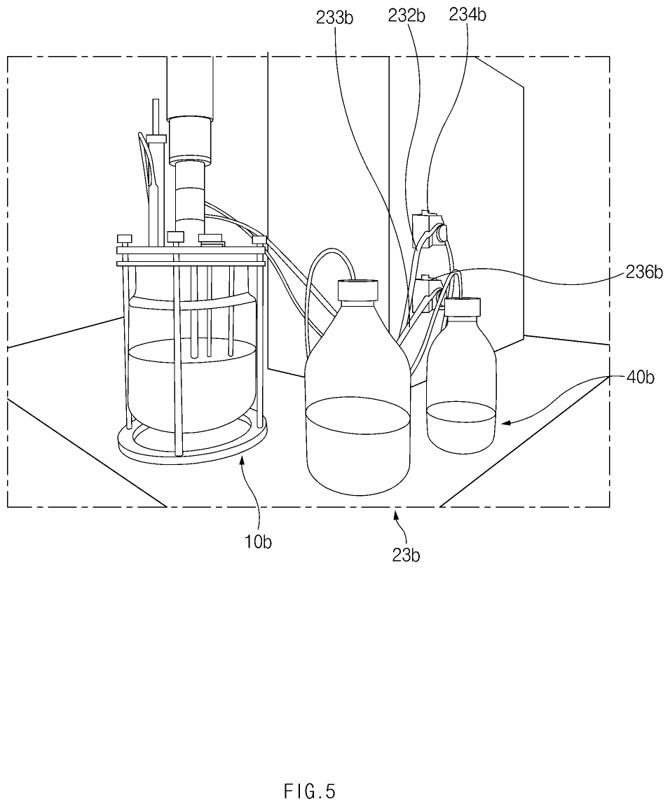

FIG. 4 is a view schematically illustrating a bioreactor system 1b according to another exemplary embodiment of the present invention. FIG. 5 is a view illustrating a state in which the bioreactor system 1b according to another exemplary embodiment of the present invention is realized.

The bioreactor system 1b according to another exemplary embodiment of the present invention is different from the bioreactor system 1 according to an exemplary embodiment in that the bioreactor system 1b does not have the separate treatment tank 21 and the culture medium exchange part 22. Other differences will be described below, and the same as described in the bioreactor system 1 according to an exemplary embodiment of the present invention may be applied to other components that are not described below.

The bioreactor system 1b according to another exemplary embodiment of the present invention includes a bioreaction unit 10b and a treatment unit 20b. The treatment unit 20b includes a dialyzer 23b that is a waste product removal part. The dialyzer 23 may be directly connected to the reaction tank 11b. Accordingly, the removal member 235b may be positioned in the reaction tank 11b, and a buffer tank 231b outside the reaction tank 11b and the removal member 235b may be connected to a first buffer transfer conduit 232b and a second buffer transfer conduit 233b. A first pump 234b may be disposed in the first buffer transfer conduit 232b, and a second pump 236b may be disposed in the second buffer transfer conduit 233b.

The bioreactor system 1b may include a feeding unit 40b capable of supplying nutrients to the reaction tank 11b. The feeding unit 40b may include a nutrient tank 41b, a nutrient conduit 42b, and a nutrient pump 43b as described above.

The processor may determine whether it is necessary to remove waste products. If it is determined that the removal of waste products is necessary, the processor may control the dialyzer 23b to remove the waste products.

The processor may determine whether the removal of waste products is necessary on the basis of the pH value of the culture medium in the reaction tank 11b obtained by a pH obtaining part 14b, and thus control the operation of the pump parts 234b and 263b. The processor may determine whether the pH value obtained by the pH obtaining part 14b is within a predetermined allowable pH range, and may determine that the removal of waste products is necessary if the pH value is out of the range. If the obtained pH value is less than the allowable pH range, the processor may increase the rotation rate of the pump parts 234b and 236b to increase the dialysis speed, thereby increasing the pH value. If the obtained pH value is greater than the allowable pH range, the processor may decrease the rotation rate of the pump parts 234b and 236b to slow down the dialysis speed, thereby decreasing the pH value. Therefore, an appropriate pH is always maintained, and thus the cells may easily grow.

The processor may control the operation of a gas inlet part 13b on the basis of the dissolved oxygen amount value of the culture medium in the reaction tank 11b obtained by the oxygen amount obtaining part 15b. The processor may introduce the gas into the reaction tank 11b through the gas inlet part 13b so that the dissolved oxygen amount value obtained by the oxygen amount obtaining part 15b reaches a reference oxygen amount. That is, according to the obtained dissolved oxygen amount value, the amount of gas flowing through the gas inlet part 13b may be determined. Therefore, an appropriate dissolved oxygen amount is always maintained, and thus the cells may easily grow.

Heretofore, even though all components configuring the exemplary embodiments of the present invention are described to be combined as one unit or to operate as a combination thereof, the present invention is not limited to these embodiments. That is, within the scope of the present invention, all components may be optionally combined to one or more thereof to operate as a combination. Also, the terms “comprise,” “configure” and/or “have” mean the presence of stated components, unless otherwise stated herein, but do not preclude the presence thereof and should be construed to further include other components. Unless otherwise defined, all terms including technical or scientific terms used herein have the same meaning as commonly understood by those skilled in the art to which the present invention pertains. General terms that are defined in a dictionary shall be construed to have meanings that are consistent in the context of the relevant art, and will not be interpreted as having an idealistic or excessively formalistic meaning unless clearly defined in the present application.

Accordingly, it is possible to easily grow cells as well as easily recycle the culture medium.

While the present invention has been shown and described in connection with the exemplary embodiments, it will be apparent to those skilled in the art that modifications and variations can be made without departing from the spirit and scope of the invention as defined by the appended claims. The exemplary embodiments should be considered in descriptive sense only and not for purposes of limitation, and do not intend to limit technical scopes of the present invention. Therefore, the scope of the invention is defined not by the detailed description of the invention but by the appended claims, and all differences within the scope will be construed as being included in the present invention.

What is claimed is:

1. A bioreactor system comprising:
a bioreaction unit configured to accommodate a culture medium; and
a treatment unit configured to recycle the culture medium transferred from the bioreaction unit,
wherein the treatment unit comprises:
a treatment tank configured to accommodate the culture medium transferred from the bioreaction unit;
a culture medium exchange part configured to exchange the culture medium between the bioreaction unit and the treatment tank; and
a waste product removal part configured to remove waste products contained in the culture medium accommodated in the treatment tank.

2. The bioreactor system of claim 1, wherein the waste product removal part comprises:
a buffer tank configured to accommodate a buffer solution; and
a removal module that is partially positioned in the treatment tank so that the buffer solution accommodated in the buffer tank is circulated through inside of the removal module and the waste products are transferred from the culture medium accommodated in the treatment tank to the buffer solution.

3. The bioreactor system of claim 2, wherein the removal module comprises:
a removal member which is positioned inside the treatment tank and has a semi-permeable membrane;
a first buffer transfer conduit configured to transfer the buffer solution from the treatment tank to the removal member; and
a second buffer transfer conduit configured to transfer the buffer solution from the removal member to the treatment tank.

4. The bioreactor system of claim 1, wherein the bioreaction unit comprises a reaction tank configured to accommodate the culture medium, and
the culture medium exchange part comprises a first exchange conduit configured to transfer the culture medium from the treatment tank to the reaction tank, a second exchange conduit configured to transfer the culture medium from the reaction tank to the treatment tank, and an exchange pump disposed in the first exchange conduit to pump the culture medium.

5. The bioreactor system of claim 4, wherein the bioreaction unit further comprises an agitator configured to stir the culture medium accommodated in the reaction tank, and a gas inlet part configured to introduce a gas into the reaction tank, and
the bioreactor system further comprises a processor electrically connected to the bioreaction unit and the culture medium exchange part,
wherein the processor operates the gas inlet part after stopping the operation of the agitator so that the culture medium moves through the second exchange conduit.

6. The bioreactor system of claim 5, wherein the processor operates the gas inlet part after a predetermined waiting time passes after stopping the operation of the agitator.

7. The bioreactor system of claim 4, wherein the culture medium exchange part further comprises an exchange valve which is disposed in the second exchange conduit to control the opening and closing of the second exchange conduit.

8. The bioreactor system of claim 1, wherein the bioreaction unit comprises a reaction tank configured to accommodate the culture medium, and a pH obtaining part configured to obtain a pH of the culture medium accommodated in the reaction tank,
the bioreactor system further comprises a processor electrically connected to the bioreaction unit and the treatment unit,
wherein the processor controls the operation of the culture medium exchange part or the waste product removal part on the basis of the pH value obtained by the pH obtaining part.

9. The bioreactor system of claim 1, wherein the bioreaction unit comprises a reaction tank configured to accommodate the culture medium, and an oxygen amount obtaining part configured to obtain a dissolved oxygen amount of the culture medium accommodated in the reaction tank,
the bioreactor system further comprises a processor electrically connected to the bioreaction unit and the treatment unit,
wherein the processor controls the operation of the culture medium exchange part or the waste product removal part on the basis of the dissolved oxygen amount value obtained by the oxygen amount obtaining part.

10. The bioreactor system of claim 1, wherein the treatment unit further comprises an aeration part configured to provide air to the culture medium accommodated in the treatment tank.

11. The bioreactor system of claim 1, further comprising a harvesting unit connected to the bioreaction unit to receive the culture medium accommodated in the bioreaction unit.

12. A cell culture method comprising:
transferring a culture medium accommodated in a reaction tank to a treatment tank;
removing waste products contained in the culture medium accommodated in the treatment tank; and
transferring, to the reaction tank, the culture medium from which the waste products are removed.

13. The cell culture method of claim 12, wherein the removing of the waste products comprises transferring the waste products from the culture medium to the buffer solution using a semi-permeable membrane by diffusion due to a concentration difference.

14. The cell culture method of claim 12, further comprising stirring the culture medium accommodated in the reaction tank, and stopping the stirring of the culture medium accommodated in the reaction tank before transferring the culture medium accommodated in the reaction tank to the treatment tank.

15. The cell culture method of claim 14, wherein the transferring of the culture medium accommodated in the reaction tank to the treatment tank is performed after a predetermined waiting time passes after the stirring of the culture medium accommodated in the reaction tank is stopped.

16. The cell culture method of claim 12, wherein the transferring of the culture medium, from which the waste products are removed, to the reaction tank is performed while the culture medium accommodated in the reaction tank is stirred.

17. The cell culture method of claim 12, wherein the transferring of the culture medium accommodated in the reaction tank to the treatment tank comprises:
introducing a gas into the reaction tank; and
transferring the culture medium from the reaction tank to the treatment tank by an increased pressure through an exchange conduit which connects the reaction tank and the treatment tank.

18. The cell culture method of claim 12, further comprising providing air to the culture medium accommodated in the treatment tank.
